(19) United States
(12) Patent Application Publication
(10) Pub. No.: US 2005/0153932 A1
(43) Pub. Date: Jul. 14, 2005

(54) Controlled muscle relaxation

(76) Inventors: Erik Dagobert Sprengers, Eindhoven (NL); Antonius Helena Adolf Bom, Livingston (GB); Mattheus Paulus van Iersel, Hoogezand-Sappemeer (NL)

Correspondence Address: AKZO NOBEL PHARMA PATENT DEPARTMENT, PO BOX 318, MILLSBORO, DE, 19966, US

(21) Appl. No.: 10/961,916

(22) Filed: Oct. 8, 2004

Related U.S. Application Data
(60) Provisional application No. 60/509,719, filed on Oct. 8, 2003.

(51) Int. Cl. A61K 31/724

(52) U.S. Cl. 514/58

(57) ABSTRACT

In general, embodiments of the present invention provide a method of applying controlled neuromuscular block in a surgical patient until the end of the surgical procedure comprising the steps of (1) inducement of deep neuromuscular block by intravenous administration of an initial bolus dose of a neuromuscular blocking agent, (2) if needed, maintenance of deep neuromuscular block by intravenous application of maintenance doses of the blocking agent, and, in various embodiments, (3) reversal of the neuromuscular block by intravenous application of a bolus dose of a chemical chelator of the pertinent neuromuscular blocking agent.

### RELATED APPLICATION

This application claims priority to U.S. provisional application 60/509,719, filed on Oct. 8, 2003, titled Controlled Muscle Relaxation.

### FIELD OF THE INVENTION

The invention relates to a method of applying controlled neuromuscular block in a surgical patient until the end of case, or in an emergency patient in need of rapid tracheal intubation.

### BACKGROUND OF THE INVENTION

Neuromuscular blocking agents (NMBAs) inhibit neuromuscular transmission by occupying post-synaptic nicotinic cholinergic receptors on skeletal muscle membranes. NMBAs are routinely used in the operating room during the administration of anesthesia to facilitate endotracheal intubation and to allow surgical access to body cavities, in particular the abdomen and thorax, without hindrance from voluntary or reflex movements. NMBAs are also used in the care of critically ill patients undergoing intensive therapy, to facilitate compliance with mechanical ventilation when sedation and analgesia alone have proved inadequate, and to prevent the violent muscle movements that are associated with electroconvulsive therapy.

Based on their mechanism of action, NMBAs are divided into two categories: depolarizing and non-depolarizing NMBAs. Depolarizing neuromuscular blocking agents bind to nicotinic acetylcholine receptors (nAChRs) at the neuromuscular junction in a way similar to that of the endogenous neurotransmitter acetylcholine. They cause an initial opening of the ion channel, producing contractions known as fasciculations. However, compared to the very rapid hydrolysis of acetylcholine by acetylcholinesterases, the depolarising NMBAs are broken down relatively slowly by these enzymes. Therefore, these NMBAs bind for a much longer period to the receptor than acetylcholine, causing persistent depolarization of the end-plate and hence neuromuscular block. Succinylcholine (suxamethonium) is the best known example of a depolarizing NMBA.

Non-depolarizing neuromuscular blocking agents compete with acetylcholine for binding to muscle nAChRs, but unlike depolarizing NMBAs, they do not activate the channel. They block the activation of the channel by acetylcholine and hence prevent cell membrane depolarization, and, as a result, the muscle will become flaccid. Most of the clinically used NMBAs belong to the non-depolarizing category. These include tubocurarine, atracurium, (cis)atracurium, mivacurium, pancuronium, vecuronium and rocuronium.

At the end of surgery or a period of intensive care, a reversal agent of NMBAs is often given to the patient to assist the recovery of muscle function. Most commonly used reversal agents are inhibitors of acetylcholinesterase (AChE), such as neostigmine, edrophonium and pyridostigmine. Because the mechanism of action of these drugs is to increase the level of acetylcholine at the neuromuscular junction by inhibiting the breakdown of acetylcholine, they are not suitable for reversal of depolarizing NMBAs such as succinylcholine. The use of AChE inhibitors as reversal agents leads to problems with selectivity, since neurotransmission in all synapses (both somatic and autonomic) involving the neurotransmitter acetylcholine is potentiated by these agents. The non-selective activation of muscarinic and nicotinic acetylcholine receptors may lead to many side-effects, including bradycardia, hypotension, increased salivation, nausea, vomiting, abdominal cramps, diarrhoea and bronchoconstriction. Therefore in practice, these agents can be used only after or together with the administration of atropine (or glycopyrrolate) to antagonize the stimulating effects of acetylcholine at the muscarinic receptors in the autonomic parasympathetic neuroeffector junctions (e.g. the heart). The use of a muscarinic acetylcholine receptor (mAChR) antagonist such as atropine causes a number of side-effects, e.g., tachycardia, dry mouth, blurred vision, difficulties in emptying the bladder and may also affect cardiac conduction.

A further problem with anticholinesterase agents is that residual neuromuscular activity must be present (>10% of baseline muscle twitch activity) to allow the rapid recovery of neuromuscular function. On several occasions, administration of NMBAs can cause complete and prolonged block of neuromuscular function (“profound block”). This may happen as a result of hypersensitivity of the patient or due to an accidental overdose, but profound block will often occur shortly after administration of a bolus dose of neuromuscular blocker. At present, there is no reliable treatment to reverse such a profound or deep block. Attempts to overcome a ‘profound block’ with high doses of AChE inhibitors has the risk of inducing a “cholinergic crisis”, resulting in a broad range of symptoms related to enhanced stimulation of nicotinic and muscarinic receptors.

In International Patent Application WO 01/12202 (Akzo Nobel N.V.) the use of chemical chelators (or sequestrants) as reversal agents has been disclosed. Chemical chelators capable of forming a guest-host complex for the manufacture of a medicament for the reversal of drug-induced neuromuscular block were described. Subsequently, in International Patent Application WO 01/40316 (Akzo Nobel N.V.) specific 6-mercapto-γ-cyclodextrin derivatives were disclosed, acting through the mechanism of chemical chelation, as reversal agents for neuromuscular block induced by amino-steroidal NMBAs such as rocuronium bromide, vecuronium bromide, pancuronium bromide and rapacuronium bromide. More specifically, the sodium salt of 6-per-deoxy-6-per-(2-carboxyethyl)thio-γ-cyclodextrin, encoded Org 25969, or another pharmaceutically acceptable salt derivative thereof, was identified as a very effective chemical chelator for the steroidal neuromuscular blocking agents, and especially so for rocuronium bromide (Adam et al., J. Med. Chem 2002, 45, 1806-1816).

### SUMMARY OF THE INVENTION

The present invention provides a method of applying controlled neuromuscular block in a surgical patient until the end of the surgical procedure, i.e. until the end of case, comprising the steps of (1) inducement of deep neuromuscular block by intravenous administration of an initial bolus dose of a neuromuscular blocking agent, (2) if needed, maintenance of deep neuromuscular block by intravenous application of maintenance doses of the blocking agent, and (3) reversal of the neuromuscular block by intravenous application of a bolus dose of a chemical chelator of the pertinent neuromuscular blocking agent.

### DETAILED DESCRIPTION OF THE INVENTION

In the method of the invention a deep or profound neuromuscular block is deliberately maintained until the end of the surgical procedure, whereupon the administration of a chemical chelator (a sequestering agent) having a high affinity for the specific neuromuscular blocking agent used, will lead to rapid reversal of the neuromuscular block.

The term deep or profound neuromuscular block refers to a neuromuscular block when there is no twitch response to electrical stimulation of the ulnar nerve or other peripheral nerves.

The neuromuscular blocking agent for use in the method of the invention can be any of the clinically used neuromuscular blocking agents in combination with a chemical chelator which can effectively encapsulate the pertinent NMB used. Examples of neuromuscular blocking agents which are clinically used and which would be useful in the present invention are described by Hunter J. M. (“New neuromuscular blocking drugs”” N. Engl. J. Med. 332, 1691-1699, 1995). Examples of classes of chemical chelators are described in WO 01/12202 (Akzo Nobel N.V.). It will be understood by the skilled person that for each NMB an optimized chemical chelator has to be designed, such that the affinity between the neuromuscular blocker (guest molecule) and chelator (host molecule) is sufficiently strong, meaning an association constant in excess of 10⁶ M⁻¹.

Preferred neuromuscular blocking agents for use in the method of the present invention are the quaternary steroidal neuromuscular blocking agents, such as exemplified by rocuronium, vecuronium, pancuronium, rapacuronium, pipecuronium and the like.

These steroidal NMBA's are preferably used in combination with cyclodextrin derivatives as reversal agents, especially with the 6-mercapto-γ-cyclodextrin derivatives disclosed in WO 01/40316 (Akzo Nobel N.V.).

In an especially preferred embodiment the invention relates to methods of applying controlled neuromuscular block in a patient in need thereof using rocuronium bromide (Esmeron®; Zemuron®) in combination with the reversal agent Org 25969, i.e. the sodium salt of 6-per-deoxy-6-per-(2-carboxyethyl)thio-γ-cyclodextrin, or another pharmaceutically acceptable salt thereof.

Rocuronium bromide (Esmeron®, Zemuron®) is a member of the class of non-depolarizing, quaternary aminosteroidal neuromuscular blocking agents (muscle relaxants) which acts by competing for nicotinic cholinoceptors at the motor end-plate without causing depolarization. Other, clinically used members of the group are pancuronium and vecuronium. Rocuronium bromide acts with a fast onset and a short-to-intermediate duration of action (ED₉₅, 0.30 mg·kg⁻¹) and is in clinical use as an adjunct to general anesthesia to facilitate tracheal intubation, to provide muscle relaxation and to facilitate mechanical ventilation.

The interaction between the chemical chelator Org 25969 and rocuronium (often used as short hand for rocuronium bromide, which is marketed under the trade names Esmeron® and Zemuron®) at neutral pH and 25° C. is characterized by an association constant Kₐ of about 15.10⁶ M⁻¹ (Born et al., Angew. Chemie 2002, 114, 276-280), resulting in a very stable host-guest complex. The affinity between Org 25969 and rapacuronium is about the same, while that of vecuronium is about 9.10⁶ M⁻¹ and that of pancuronium is about 2.6.10⁶ M⁻¹. Tests in several animal models including guinea pigs, cats and monkeys showed Org 25969 to be able to rapidly reverse rocuronium-induced block.

In many surgical interventions full neuromuscular relaxation of the patient is an absolute requirement; in others it is a great improvement to the operating conditions, resulting in better, safer and faster surgical procedures. However full relaxation goes at the expense of a long and, on an individual basis, unpredictable recovery time. For rocuronium the relationship between dose and time to maximum block and clinical duration of action in a patient can be seen from the following table:

| Rocuronium dose | | Time to >80% | Time to maximum | | Clinical* | |
|---|---|---|---|---|---|---|
| mg.kg − 1 | | block (min) | block (min) | | duration (min) | |
| 0.45 | (n = 50) | 1.3 (0.8-6.2) | 3.0 | (1.3-8.2) | 22 | (12-31) |
| 0.6 | (n = 142) | 1.0 (0.4-6.0) | 1.8 | (0.6-13.0) | 31 | (15-85) |
| 0.9 | (n = 20) | 1.1 (0.3-3.8) | 1.4 | (0.8-6.2) | 58 | (27-111) |
| 1.2 | (n = 18) | 0.7 (0.4-1.7) | 1.0 | (0.6-4.7) | 67 | (38-160) |

[*clinical duration is defined as the time until return to 25% of control T1.]

Because of the long recovery times, it is current practice that towards the end of a surgical intervention the anesthesiologist needs to find a compromise between the quality of the surgical conditions and the time to full recovery after the end of the surgical procedure, which in its turn is a factor determining operating theatre throughput. For instance the dose of inhalation anesthetic agent which potentiates the action of rocuronium may be temporarily increased, or an extra dose of opiate is given to the same effect (“balanced anesthesia”), while further neuromuscular blocker is not administered. These measures come with increased risk such as respiratory depression and/or depression of cardiac function.

A significant advantage of the method of the present invention is that there is no need to take such compromising measures and thus no need to cope with the increased risks. Maintaining deep block until the surgical procedure has been fully completed also provides for better operating conditions, leading to improved quality of stitching, decreased chance of bleeding as a result of bad stitches, shortening of the surgical procedure and better wound healing.

In one aspect of the present invention a method is provided to apply controlled neuromuscular block in a patient, comprising (1) inducement of neuromuscular block by intravenous administration of an initial bolus dose of 0.6-2.1 mg·kg⁻¹ (corresponding to 2-7×ED₉₀) of rocuronium bromide and (2) reversal of the neuromuscular block by intravenous application of a bolus dose of 1.0-25 mg·kg⁻¹ of 6-per-deoxy-6-per-(2-carboxyethyl)thio-γ-cyclodextrin sodium salt. If the neuromuscular block is to be maintained beyond the clinical duration of the initial bolus administration of rocuronium bromide the neuromuscular block is retained during surgery by maintenance doses of 0.1-0.3 mg·kg⁻¹ of rocuronium bromide administered at 10% recovery of control T1, i.e. as early as the appearance of 10% of control T1, or by infusion of rocuronium.

Since all administrations of the neuromuscular blocker can be antagonized to full recovery within 5 minutes, the method of the invention allows the anesthesiologist to maintain a complete and/or optimal neuromuscular block throughout and until the end of the actual surgical procedure [“full relaxation till the last stitch”].

Another advantageous aspect of the present invention is that the incidence of residual neuromuscular blockade, which is still reported to present a problem even after short surgical procedures (Debaene et. al. Anesthesiology 98, 1042-1048, 2003), will be much reduced or even be absent as a result of the effective complexation in vivo of rocuronium bromide by Org 25969.

Another aspect of the invention relates to a method of applying neuromuscular block in an emergency patient in need of rapid tracheal intubation.

The vocal cords play an important role in the protection of the airway against foreign bodies. The vocal cords are equipped with an extremely strong reflex closing mechanism in case of intrusion of the larynx with corpora aliena. When during an intubation procedure of the trachea the vocal cords are (partly) relaxed, such defense mechanisms are impaired and the patient is at risk of contaminating the lower respiratory tract with, for example, stomach contents. In elective surgery patients appear well prepared in the operating theatre, in order to minimize surgical and anesthesiological risks. In particular patients will have an empty stomach.

Rapid sequence induction is a procedure of rapid intubation required for patients that do not have an empty stomach, such as emergency patients (trauma, unscheduled caesarian section, etc.). In these procedures very rapid intubation is required.

For this indication, two muscle relaxants are on the market: suxamethonium and rocuronium.

Although obsolete from a side effect point of view, suxamethonium is still widely used for endotracheal intubation because of its neuromuscular blocking profile. It has a very fast onset (˜1 minute) and a very short duration of action (˜5-8 minutes). For rapid sequence induction this combines the advantages of fast onset and also it provides safety in case intubation is not successful: in this case the patient will be able to breathe spontaneously and have a secured airway shortly after the administration of the muscle relaxant.

Rocuronium is indicated for rapid sequence induction, using doses of 0.6-1.2 mg·kg⁻¹, the higher doses giving a faster onset. At 1.2 mg·kg⁻¹ time to maximum block is 1.0 minute (range: 0.6-4.7 minutes). However, this is associated with a clinical duration of 67 minutes (range: 38-160 minutes). In case of a failed intubation there is no means to antagonize the effect of the neuromuscular blocker and secure the patient's airway. From the point of view of it's side effect profile rocuronium is far superior to suxamethonium. However, the long duration of action of rocuronium and the lack of possibilities to antagonize neuromuscular block are a disadvantage.

The present invention completely abolishes these disadvantages. The new use consists of rapid sequence induction with a fixed dose of rocuronium (for example 1.0 mg·kg⁻¹) and, in case of failed intubation (for example after 2 failed attempts and/or 3 minutes after administration of rocuronium) antagonizing the neuromuscular blocking effect with Org 25969 after a certain period of time. In addition, in case of a successful intubation, the neuromuscular blocking action of rocuronium can be antagonized at the end of the procedure, using Org 25969.

Advantages are improved safety in case of “cannot intubate, cannot ventilate” situations and improved safety because of better side effect profile of rocuronium bromide in comparison to suxamethonium. Thus in such situations where intubation and ventilation in a paralyzed patient is not possible Org 25969 can be used to effect “rescue reversal”.

The invention is illustrated in the following Examples:

General Methods:

Peripheral nerve stimulation is being used in the operating room by anesthesiologists, as well as in the Intensive Care Units (ICU), to titrate the dose of neuromuscular blocking agent to match the degree of paralysis needed.

A simple method of monitoring neuromuscular function is the train-of-four (TOF) technique using an acceleromyograph (TOF Watch®). Four electrical stimuli each 0.5 seconds apart (and for instance applied at 12-15 seconds intervals) are applied along the course of the ulnar nerve in the arm. Stimulation of the ulnar nerve causes contraction of the adductor pollicis muscle and movement of the thumb. Response can be noted visually or, more accurately, by placing a finger lightly on the surface of the hand over the muscle to feel the contraction. A baseline response check is done to ensure that there are four muscle twitches in response to the four stimuli. Normal baseline and full recovery is indicated by four twitches of equal strenght. As the number of acetylcholine receptors occupied by NMBAs increases, the number of twitches decreases, no twitches indicating total paralysis. The presence of three twitches indicates that 75-80% of the receptors are blocked, two twitches indicate 80-90% block and one twitch out of four 90% blockade.

The terms profound neuromuscular block(ade) or deep block refer to a neuromuscular block when there is no twitch response to stimulation of the ulnar nerve.

Definitions:

T₁: first twitch: amplitude of the first response to TOF stimulation, expressed as percentage of control T₁ (%).
T₄/T₁ ratio: the ratio of T₄ over T₁ (within one TOF stimulus) expressed as decimals. At complete recovery the T₄/T₁ ratio is 1.0.
Recovery time until TOF 0.9: the time from the start of administration of reversal agent (=chemical chelator) or placebo until recovery of the T₄/T₁ ratio to 0.9, either during spontaneous or induced recovery (hr min sec).
TOF: train-of-four; four consecutive square wave supra-maximal stimuli of 0.2 ms duration delivered at a frequency of 2 HZ (repeated e.g. every 15 seconds).
Onset time: time period between start of the administration of the neuromuscular blocking agent and either (a) 95% depression of the 1^{st} twitch or, if this is not reached (b) the first of three consecutive twitches with the same or increasing amplitude.
ED₅₀: dose at which the effect is 50% of the maximum effect.

### EXAMPLE 1

Org 25969-Induced Reversal of Rocuronium-Induced Profound Block of M. Gastrocnemius Contractions in Anesthetised Guinea Pigs.

A: Assay.

Male healthy mature guinea pigs were anesthetised with 30 mg·kg⁻¹ pentobarbitone and 900 mg·kg⁻¹ urethane i.p. The guinea pigs were artificially ventilated with room air (4.5-5.5 ml; 50-60 strokes/min). Catheters were placed in both jugular veins and a third catheter was placed in the carotid artery for continuous monitoring of arterial blood pressure and the taking of blood samples for blood gas analysis.

Heart rate was derived from the blood pressure signal.

Body temperature was maintained at 37-38° C. using an electrical heating pad.

The gastrocnemius muscle was dissected free from surrounding muscles and set up for recording twitch tension using a force transducer. A resting tension of 250-300 mN was applied to the muscle. A stimulation electrode was placed around the sciatic nerve, which was crushed centrally to the electrode to prevent reflex responses. The gastrocnemius muscle was stimulated indirectly via the sciatic nerve with rectangular pulses of 0.5 ms duration at 10 s (0.1 Hz) intervals at a supra maximal voltage using a Grass S88 Stimulator.

Animals were allowed to stabilise for −30 min before the start of the experiment. Firstly, the 1×ED₉₀ blocking dose of rocuronium was established in each animal followed by complete spontaneous recovery of muscle contractions. After at least 30 minutes, all animals received a 10×ED₉₀ blocking dose of rocuronium by intravenous bolus injection. One minute after bolus injection of rocuronium, the animals received either 2300 nmol·kg⁻¹ Org 25969 in saline or an equivalent volume of saline. Time to 25%, 50%, 75% and 90% recovery of twitch height was measured from the moment of injection of Org 25969 or saline, respectively.

Rocuronium bromide was dissolved in a pH 4.0 citric acid/phosphate/mannitol buffer. Org 25969 was dissolved in saline.

B: Results (See Table 1).

No differences in blocking doses, maximum block and spontaneous recovery indices were observed between the two groups after the 1×ED₉₀ dose.

Bolus injection of a 10×ED₉₀ dose caused complete block in both groups.

In the group subsequently treated with saline, a very slow recovery was observed (e.g. 25-75% recovery time of 15.9 min and a time to 90% recovery of 78.3 min). In contrast, the animals of the other group receiving Org 25969 recovered very fast (25-75% recovery time of 1.3 min and a time to 90% recovery of 8.5 min).

The results in Table 1 demonstrate that Org 25969 is very effective in reversing profound block induced by rocuronium. No signs of re-curarisation were observed in the hour following Org 25969 administration. No important changes in haemodynamic parameters were observed after administration of Org 25969.

TABLE 1
Neuromuscular blocking parameters after an i.v. bolus
injection of a 1× ED₉₀ dose of rocuronium in
anaesthetised guinea pigs, followed by spontaneous
recovery, and a bolus injection of a 10× ED₉₀ dose
of rocuronium, followed by i.v. injection of either
saline or 2300 nmol · kg⁻¹ Org 25969.

| | group 1 | group 2 |
|---|---|---|
| 1× ED₉₀ dose (nmol · kg⁻¹): | 178 ± 22 | 169 ± 15 |
| max block (%): | 89.7 ± 2.4 | 91.0 ± 2.5 |
| onset time (min): | 2.6 ± 0.3 | 2.1 ± 0.3 |
| time to 25% recovery (min): | 5.1 ± 0.7 | 3.8 ± 0.5 |
| time to 90% recovery (min): | 10.7 ± 1.5 | 8.7 ± 1.4 |
| 25-75% recovery index(min): | 3.5 ± 0.4 | 2.3 ± .5 |
| max recovery (%): | 101.1 ± 1.3 | 101.4 ± 2.8 |
| after (min): | 15.1 ± 2.1 | 12.5 ± 1.7 |
| 10× ED₉₀ dose (nmol · kg⁻¹): | 1783 ± 216 | 1692 ± 147 |
| max block (%): | 100.0 ± 0.0 | 100.0 ± 0.0 |
| onset time (min): | 2.0 ± 0.8 | 1.7 ± 0.5 |
| | | Org 25969 |
| bolus injection of | saline | (2300 nmol · kg⁻¹) |
| time to 25% recovery (min): | 47.5 ± 7.0 | 3.7 ± 0.4 |
| time to 90% recovery (min): | 78.3 ± 15.2 | 8.5 ± 2.2 |
| 25-75% recovery index(min): | 15.9 ± 4.0 | 1.3 ± 0.4 |

Mean ± SEM; n = 6 in each group.

### EXAMPLE 2

Effect of Org 25969 on Neuromuscular Block of M. Adductor Pollicis Contractions Induced by Bolus Administration of a 5×ED₉₀ dose of Rocuronium and Vecuronium in Anaesthetised Rhesus Monkeys During Train-of-Four (TOF) Stimulation of the Ulnar Nerve.

A: Assay.

Female Rhesus monkeys were starved overnight and sedated with 10 mg·kg⁻¹ ketamine i.m., followed by i.v. injection of 25 mg·kg⁻¹ pentobarbitone sodium and subsequent infusion of 5-10 mg·kg⁻¹hr⁻¹. The animals were intubated and ventilated with a mixture of oxygen and nitrous oxide (2:3), using a ventilator. The expired CO₂ level was measured using a capnograph. The tidal volume was adjusted to obtain a CO₂ level of approximately 4.5%. Oxygen saturation was measured via a probe on the earlobe using a pulse oxymeter. Blood pressure was measured non-invasively with a cuff placed on the tail. The ECG was recorded and heart rate was measured. Rectal temperature was measured and body temperature was kept at 37-38° C. using a heating blanket. The right hand and right arm of the animal were fixed to a frame using surgical tape. The thumb was connected to a force transducer. A pre-load of 200-500 mN was applied. Two needle electrode were placed near the N. ulnaris and the nerve was stimulated with train-of-four pulses (pulse duration: 2 ms, train-frequency: 0.07 Hz). At the end of the experiment, the animals were allowed to recover from anesthesia. Appropriate recovery care was given to all animals. Two groups of monkeys were used to assess the reversal effects of Org 25969 against the neuromuscular blocking agents rocuronium (Esmeron®/Zemuron®) and vecuronium (Norcuron®). In the first group, animals initially received an intravenous bolus injection of a 1×ED₉₀ dose of rocuronium bromide to induce neuromuscular block, followed by spontaneous recovery of the twitches. After complete recovery of the twitches, each group received a second and larger 5×ED₉₀ dose of rocuronium followed by spontaneous recovery in one subgroup, whereas the other subgroup received an intravenous bolus dose of 1150 nmol·kg⁻¹ Org 25969 as soon as all twitches had disappeared. In the vecuronium groups of animals, the same protocol was used, but rocuronium was replaced by vecuronium. Rocuronium bromide and vecuronium bromide were dissolved in a citric acid/phosphate/mannitol buffer (pH 4.0). Org 25969 was dissolved in saline.

B: Results (See Table 2 and 3)

In both subgroups, receiving the 1×ED₉₀ bolus dose, rocuronium caused a similar depth of neuromuscular block. The spontaneous recovery indices were also similar in both subgroups. Not surprisingly, a 5×ED₉₀ bolus dose of rocuronium caused complete neuromuscular block. Administration of 1150 nmol·kg⁻¹ Org 25969 i.v. caused rapid reversal of rocuronium-induced neuromuscular block. All recovery indices were significantly shorter than those observed during spontaneous recovery from a 5×ED₉₀ dose. Interestingly, when the recovery indices of a 5×ED₉₀ dose were compared with those of a 1×ED₉₀ dose in the same experiment, all recovery indices, except the 25-75% recovery index and the time interval between a TOF ratio of 0.50 and 0.90, were significantly shorter than after an 1×ED₉₀ dose. This demonstrates the remarkable reversal properties of Org 25969. This also shows that the Org 25969-induced recovery seems to occur in a parallel fashion to the spontaneous recovery.

The 1×ED₉₀ bolus dose of vecuronium caused a similar depth of neuromuscular block in both subgroups of animals. The spontaneous recovery indices in the two subgroups were likewise not significantly different. Not surprisingly, a 5×ED₉₀ bolus dose of vecuronium caused complete neuromuscular block, followed by a slower recovery. Administration of 1150 nmol·kg⁻¹ Org 25969 i.v. caused an initial improvement of the time to 25% and 50% recovery values, but the recovery slowed down, resulting in a non-significant improvement in the remaining recovery parameters. The 25-75% recovery index and the time interval between a TOF ratio of 0.50 and 0.90 were prolonged after administration of Org 25969.

In contrast to the results with rocuronium, the recovery from a 5×ED₉₀ dose of vecuronium after administration of Org 25969 seems to occur in a non-parallel fashion.

These data clearly show that, the dose of 1150 nmol·kg⁻¹ Org 25969 is more effective against a 5×ED₉₀ dose of rocuronium than against a similar multiple of the ED₉₀ concentration of vecuronium.

TABLE 2
Effect of 1× ED₉₀ and 5× ED₉₀ dose of rocuronium and of the 5× ED₉₀ dose
followed by 1150 nmol · kg⁻¹ Org 25969 on M. adductor
pollicis contractions in anaesthetised Rhesus monkeys during train-of-four
stimulation.

| | | | 5× ED₉₀ + |
|---|---|---|---|
| | 1× ED₉₀ | 5× ED₉₀ | Org 25969 |
| rocuronium dose (nmol · kg⁻¹): | 160 ± 4 | 800 ± 20 | 800 ± 20 |
| block (%): | 93.3 ± 1.9 | 100.0 ± 0.0 | 100.0 ± 0.0 |
| onset time (min): | 1.2 ± 0.0 | 0.7 ± 0.2 | 0.5 ± 0.1 |
| time to 25% recovery of T1 (min): | 4.5 ± 0.6 | 18.5 ± 2.1 | 3.8 ± 0.7 |
| time to 90% recovery of T1 (min): | 9.6 ± 0.9 | 27.7 ± 3.2 | 9.5 ± 1.9 |
| 25-75% recovery index (min): | 3.3 ± 0.4 | 6.0 ± 0.6 | 3.1 ± 0.8 |
| time to TOF ratio 0.90 (min): | 8.4 ± 0.9 | 28.2 ± 3.4 | 7.9 ± 1.8 |
| 0.50-0.90 TOF ratio interval (min): | 2.6 ± 0.2 | 6.9 ± 0.8 | 4.0 ± 0.9 |

Mean ± SEM; n = 4
T1 = first twitch induced by train-of-four (TOF) stimulation.
25-75% recovery index: time between 25% and 75% recovery of T1.

TABLE 3
Effect of 1× ED₉₀ and 5× ED₉₀ dose of vecuronium and of the 5× ED₉₀ dose
followed by 1150 nmol · kg⁻¹ Org 25969 on M. adductor
lollicis contractions in anaesthetised Rhesus monkeys during train-of-four
stimulation.

| | | | 5× ED₉₀ + |
|---|---|---|---|
| | 1× ED₉₀ | 5× ED₉₀ | Org 25969 |
| vecuronium dose (nmol/kg): | 14 ± 1 | 70 ± 4 | 70 ± 4 |
| block (%): | 91.5 ± 1.7 | 100.0 ± 0.0 | 100.0 ± 0.0 |
| onset time (min): | 4.3 ± 0.6 | 1.0 ± 0.1 | 1.0 ± 0.2 |
| time to 25% recovery of T1 (min): | 8.6 ± 1.0 | 28.6 ± 2.8 | 9.7 ± 1.9 |
| time to 90% recovery of T1 (min): | 18.1 ± 1.9 | 44.0 ± 4.6 | 35.8 ± 7.1 |
| 25-75% recovery index (min): | 5.4 ± 0.8 | 9.6 ± 2.0 | 16.1 ± 3.7 |
| time to TOF ratio 0.90 (min): | 17.2 ± 1.6 | 49.0 ± 4.7 | 48.6 ± 8.3 |
| 0.50-0.90 TOF ratio interval (min): | 4.9 ± 0.5 | 11.8 ± 1.3 | 24.2 ± 3.9 |

Mean ± SEM; n = 4.
T1 = first twitch induced by train-of-four (TOF) stimulation.
25-75% recovery index: time between 25% and 75% recovery of T1.

### EXAMPLE 3

Org 25969 as Reversal Agent of Deep Block Induced by Rocuronium in Healthy Male Volunteers.

10 Volunteers, which were enrolled in a phase 1 trial with the objective to investigate the safety, pharmacokinetics and efficacy of Org 25969, received general anesthesia using i.v. administration of remifentanil followed by propofol (Target controlled Infusion—Diprifusor®) for induction and maintenance. After induction of anesthesia and set-up of the TOF-Watch®SX subjects received a i.v. bolus dose of 0.6 mg·kg⁻¹ rocuronium bromide (Esmeron®) given by hand over 5 seconds. Between the administration of rocuronium and the administration of Org 25969, a laryngeal mask was applied or tracheal intubation was performed to ventilate the subjects. At 3 minutes after rocuronium bromide administration (i.e. deep block) either placebo (commercially physiological salt solution, 0.9% sodium chloride) or Org 25969 (25 mg/ml) were given as a bolus dose into a fast running infusion in the forearm. Six dose levels of Org 25969 were given to these 10 volunteers. Neuromuscular block was measured using the TOF-Watch-SX® acceleromyograph for at least 90 minutes after administration of Org 25969 or placebo.

TABLE 4
time from administration of Org 25969 until recovery
of neuromuscular block to TOF ratio to 0.9:

| | Dose Org 25969 in mg · kg⁻¹ | | | | | | |
|---|---|---|---|---|---|---|---|
| | placebo | 0.1 | 0.5 | 1.0 | 2.0 | 4.0 | 8.0 |
| Volunteers (n) | 10 | 1 | 1 | 2 | 2 | 2 | 2 |
| Time to TOF ratio 0.9 (min) | 35 to 69 | 43 | 71 | 23 | 13 | 2.5 | 1.0 |
| | | | | 31 | 17 | 3.2 | 1.2 |

In the two volunteers to whom 8 mg·kg−1 of Org 25969 was given, the train-of-four (TOF) ratio returned to 0.9 within 2 minutes after administration of Org 25969 (see Table 4). After complete recovery occurred, twitches were monitored for a period of one hour, showing no signs of re-curarisation.

(Gijsenbergh et al., Anesthesiology, 96, A-1008, 2002)

1. A method of applying controlled neuromuscular block in a surgical patient until the end of case comprising the steps of (1) inducement of deep neuromuscular block by intravenous administration of an initial bolus dose of a neuromuscular blocking agent, (2) if needed, maintenance of deep neuromuscular block by intravenous application of maintenance doses of the blocking agent, and (3) reversal of the neuromuscular block by intravenous application of a bolus dose of a chemical chelator of the pertinent neuromuscular blocking agent.

2. The method of claim 1, wherein the neuromuscular blocking agent is a steroidal neuromuscular blocking agent and the chemical chelator is a cyclodextrin derivative.

3. The method of claim 2, wherein the steroidal neuromuscular blocking agent is rocuronium bromide and the chemical chelator is of 6-per-deoxy-6-per-(2-carboxyethyl)thio-γ-cyclodextrin sodium salt (Org 25969).

4. The method of claim 3, comprising (1) inducement of deep neuromuscular block by intravenous administration of an initial bolus dose of 0.6-2.1 mg·kg⁻¹ of rocuronium bromide, (2) if needed, maintenance of deep neuromuscular block by intravenous application of maintenance doses of 0.1-0.3 mg·kg⁻¹ of rocuronium bromide administered at 10% recovery of control T1, and (3) reversal of the neuromuscular block by intravenous application of a bolus dose of 1.0-25 mg·kg⁻¹ of 6-per-deoxy-6-per-(2-carboxyethyl)thio-γ-cyclodextrin sodium salt (Org 25969).

5. The method according to claim 4, wherein the initial bolus dose of rocuronium bromide is 0.6-1.2 mg·kg⁻¹.

6. The method according to any one claims 3-5, wherein the reversal of neuromuscular block is by intravenous application of a bolus dose of 4.0-12.0 m mg·kg⁻¹ 6-per-deoxy-6-per-(2-carboxyethyl)thio-γ-cyclodextrin sodium salt (Org 25969)

7. The method of applying neuromuscular block in an emergency patient in need of rapid tracheal intubation, comprising (1) inducement of deep neuromuscular block by intravenous administration of a bolus dose of 0.6-2.1 mg·kg⁻¹ of rocuronium bromide, and wherein either in case of a failed intubation or at the end of the emergency procedure the neuromuscular block is reversed by intravenous application of a bolus dose of 1.0-25 mg·kg⁻¹ of 6-per-deoxy-6-per-(2-carboxyethyl)thio-γ-cyclodextrin sodium salt (Org 25969).
